(19) United States
(12) Patent Application Publication
(10) Pub. No.: US 2004/0029104 A1
(43) Pub. Date: Feb. 12, 2004

(54) Method of detecting and quantifying endotoxins

(75) Inventors: David W. Stanley, Lincoln, NE (US); Rosevelt L. Pardy, Lincoln, NE (US)

Correspondence Address: Michael J. Gross, SHOOK, HARDY & BACON L.L.P., One Kansas City Place, 1200 Main Street, Kansas City, MO, 64105-2118, US

(73) Assignee: BOARD OF REGENTS OF UNIVERSITY OF NEBRASKA

(21) Appl. No.: 10/636,944

(22) Filed: Aug. 7, 2003

Related U.S. Application Data
(62) Division of application No. 09/318,358, filed on May 25, 1999.

(51) Int. Cl. C12Q 1/00

(52) U.S. Cl. 435/4

(57) ABSTRACT

The present invention relates to a method of detecting and quantifying a contaminant in a sample utilizing an insect based bioassay system. The method comprises exposing insect hemocytes, either in vivo or in vitro, to the sample to be tested followed by analyzing the cellular and biochemical behavior of the cells in the hemolymph subsequent to the exposure.

### CROSS-REFERENCE TO RELATED APPLICATIONS

This application is a divisional application that claims the benefit of U.S. application Ser. No. 09/318,358 filed on May 25, 1999, the contents of which are incorporated by reference.

### STATEMENT REGARDING FEDERALLY SPONSORED RESEARCH OR DEVELOPMENT

Not Applicable.

### BACKGROUND OF THE INVENTION

This invention relates to a method of detecting and quantifying contaminants such as whole bacterial cells, bacterial modulins, endotoxins, pathogens, and parasites, present in a solution, such as a vaccine, drug, or other biologic solution.

Biotechnology and food industries are concerned with the above-listed contaminations at all stages of manufacturing and processing. Endotoxins, such as lipopolysaccharides (LPS), are stable compounds that have biological activity whether attached or released from a bacterial membrane. Testing for these and other contaminants is standard in manufacturing processes throughout the biotechnology and food industries.

One type of contaminant of particular importance is the class known as bacterial modulins. Bacterial modulins are cellular components, including cell parts, cell molecules, and cell products that initiate or promote biological responses involving systems associated with the immune system. These bacterial modulins, together with other biochemical constituents of bacteria, comprise the antigens responsible for the immunization reactions of a vaccine. Additionally, these modulins stimulate a range of physiological responses, such as fever, and a complex of potentially lethal reactions known as sepsis. One example of this type of modulin is the lipopolysaccharide endotoxin (LPS). LPS comprises the outer membrane of gram negative bacteria. Substantial amounts of LPS, as part of the cell walls of inactivated gram negative bacteria or as an adjuvant, can be found in antibacterial vaccines. LPS is one of the most potent modulins and possesses a significant health risk. Additionally, LPS is responsible for the catastrophic release of cytokines and resulting circulatory shock associated with endotoxic sepsis caused by gram negative infections.

The presence of bacterial modulins in biologics utilized in animal and human vaccines creates a potentially lethal health risk. This risk can be measured in terms of substantial economic impacts. As such, a variety of bioassays are currently employed to detect and measure modulins in a wide variety of fluids and substances. These methods include the rabbit pyrogen assay, chick embryo lethality assay, galactose amine primed mice-lethality test, and the Limulus amoebocyte lysate assay (LAL). Of these tests, LAL is the most fiscally responsible and widely accepted bioassay.

However, despite its high specificity for LPS and great sensitivity, LAL can be problematic. For example, successful LAL analysis depends on access of the LAL reagents to LPS in a solution or suspension. Additionally, inhibiting or inactivating substances of LAL must be detected to ensure a proper analysis. For example, cationic proteins form endotoxin LPS protein complexes which impair endotoxin detection with LAL. Additionally, liposome encapsulated LPS may escape detection. Furthermore, endotoxins in some complex biological fluids shown to be pyrogenic with other tests may fail detection by LAL. An additional problem associated with LPS detection utilizing LAL is that reactivity between LPS and LAL may be attenuated by vaccine adjuvants, such as aluminum hydroxide and aluminum chloride.

There is evidence that different forms of LPS, such as the R and S form, have varying capacities to react with LAL, and that low molecular weight LPS may go undetected in biological fluids even though it retains cytokine stimulating activity. Thus, while the use and importance of LAL for endotoxin testing is widely accepted, approved, and unchallenged, the mounting experimental evidence cited above indicates that LAL may not be suited for all testing situations. Therefore, a method for the detection and quantification for contaminants in a sample is required to overcome the problems associated with the current methods of detection.

### SUMMARY OF THE INVENTION

Accordingly, it is an object of the present invention to provide a method for detecting contaminants in a sample.

Another object of the present invention is to provide a kit comprising the elements necessary for detecting and quantifying contaminants in a sample.

Another object of the present invention is to provide an article of manufacture comprising packaging material and the elements necessary for detecting and quantifying contaminants in a sample.

Accordingly, the present invention provides for a method of detecting and quantifying contaminants in a sample comprising exposing an insect hemocyte to the sample, and detecting a biochemical response.

By providing the method of the present invention, several advantages are realized. For example, trace amounts of contaminants such as a bacterial cells, bacterial modulins, endotoxins, pathogens, or parasites can be detected in a variety of samples. Additionally, by utilizing the method of the present invention the amount of contaminants in the sample can be quantitated.

Another advantage provided by the present invention is the low cost of detection.

A further advantage is the ease with which samples can be screened for contaminants.

Additional objects, advantages, and novel features of the invention will be set forth in part in a description which follows and in part will become apparent to those skilled in the art upon examination of the following, or may be learned by practice of the invention.

### BRIEF DESCRIPTION OF THE DRAWING

FIG. 1 is a two-dimensional graph illustrating a dose response curve for LPS in a tobacco hornworm system.

### DETAILED DESCRIPTION OF THE PREFERRED EMBODIMENTS

Insects do not produce specific antibodies to immunological challenges, however, they do possess sensitive and specific blood born reactions that provide processes to deal with invasive contaminants. Particularly, insects are capable of mounting rapid and decisive measures directed against contaminants. These measures involve processes initiated and sustained by the circulating hemocytes following contaminant introduction. These reactions include clotting, cell adhesion, cell aggregation, nodulation, phagocytosis, and the elaboration of specific binding proteins. For purposes of the present invention, the above-described measures are defined as the immunological response. As illustrated above, the immunological response is a series of complex biochemical reactions.

For purposes of the present invention, an insect is defined as an arthropod, class Insecta.

It has been demonstrated in several insect species that contaminants, such as whole bacteria, administered intraperitoneally are rapidly cleared from hemolymph through the formation of pigmented nodules through a process of nodulation. These nodules are masses of hemocytes that aggregate and entrap the contaminants. Within minutes of nodule formation, the nodules undergo melanization. Examination of the hemolymph of the insect subsequent to contaminant injection reveals an array of readily countable black specks or nodules adhering to the internal body wall and the internal organs of the insect. In several experiments, it has been shown that the quantity of nodules produced in response to the introduction of a particular contaminant is dependent on the dose of the contaminant.

Prior to nodulation, it has been observed that microaggregates are formed from hemocytes in the hemolymph. Like the nodules, these microaggregates can be observed when withdrawn from insects challenged by contaminants. These microaggregations are easily visible with light microscopy and have also been demonstrated to exhibit a dose dependency with respect to the amount of injected contaminant. Additionally, it has been demonstrated that both the nodulation and microaggregation can be stimulated when low doses of bacteria are used as the contaminant.

Based on the foregoing, it has been determined that an insect bioassay system is ideal for the detection of contaminants in a sample. The definition of a contaminant is any substance that causes an immunological response in an insect species selected for the insect bioassay system of the present invention. Specific examples of contaminants include bacterial cells, bacterial modulins, endotoxins, pathogens and parasites.

This system is particularly useful for the biotechnology and food industries. For example, the insect bioassay system of the present invention can be utilized for screening pharmaceuticals and food products to determine the presence of trace amounts of contaminants. This screening can be accomplished by simply injecting a sample to be tested into an insect, waiting about an hour, withdrawing a blood sample, and observing the sample under a microscope to determine the presence or absence of microaggregates. If microaggregates are present, they can be counted and compared to a range of known samples to quantitate the contaminant.

Generally, the method of the present invention utilizes the tobacco hornworm, Manduca sexta, as the insect bioassay system. However, any insect that undergoes microaggregation in response to the introduction of a contaminant can be utilized in the method of the present invention. Examples of other insects tested include Agrotis epsilon, Pseudaletia unipuncta and Zophobas atratus as illustrated in J. Insect Physiol., 1997, 43:125-133 and J. Insect Physiol., 1998, 44:157-164, which are both hereby incorporated by reference. There are several reasons why the homworm is utilized in the preferred embodiment. For example, the hornworm is about the size of an index finger, making it easy to work with; and, due to its size, there is a large volume of hemolymph available to work with. The typical hornworm contains 2 to 3 milliliters of hemolymph. One milliliter of hemolymph contains around 10-15 million cells. Additionally, the size of the hornworm allows for easy injections of a sample to be tested.

In the preferred embodiment, the method for the insect bioassay system utilizes immunologically naive tobacco hornworms. While it is not necessary to utilize the immunologically naive hornworms, their use facilitates the detecting and quantifying methods of the present invention. However, if immunologically naive hornworms are not utilized, additional control experiments are usually required. These control experiments are described in J. Insect Physiol., 1996, 42:3-12, which is hereby incorporated by reference. The naive hornworms are prepared by combining tobacco homworm eggs with about 90% alcohol for a short period, about 10 minutes. This first step surface sterilizes the homworm eggs. The now surface sterilized eggs are rinsed in sterile water. The surface sterilized eggs are then grown in a sterilized medium in a sterile environment until they are ready to be utilized in the method of the present invention. Additionally, those skilled in the art will know where to purchase or how to prepare the surface sterilized insects.

The intact living immunologically naive tobacco homworm is then injected with a sample to be tested. These injections are performed with a syringe as described in J. Insect. Physiol., 1996, 42:893-901, which is hereby incorporated by reference. Additionally, one skilled in the art would know how to perform the injections. While any type of syringe can be utilized, the syringe utilized in the preferred embodiment comprises a 26-gauge 0.5″ needle attached to a 50 ul syringe. The injections are performed by surface sterilizing the point of injection on the hornworm with 95% ethanol, followed by inserting the needle into the intersegmental suture between and just above the last two spiracles. Then, moving forward into the immediate anterior segment, the needle is kept parallel to the body wall to avoid injuring the alimentary canal, while depressing the plunger. The needle is then removed from the insect. Prior to these injections the hornworms are anesthetized by chilling on ice for about 15 minutes.

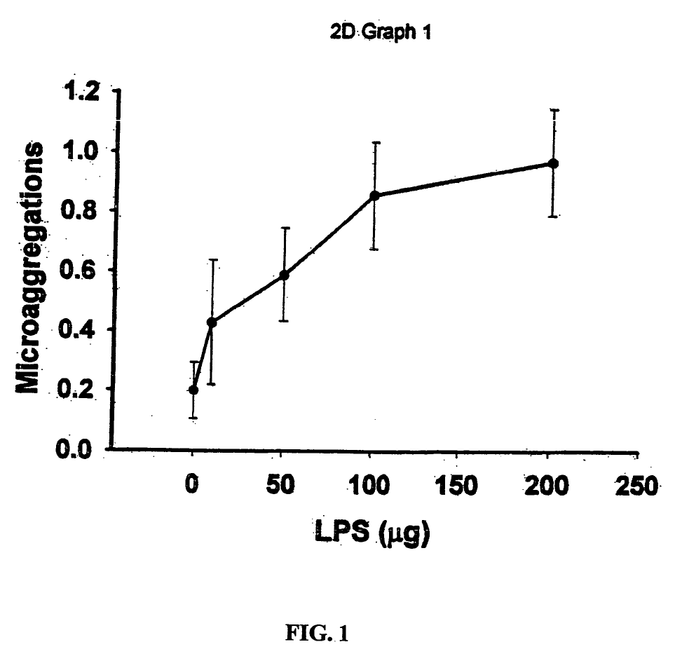

After the injection, in as little as 60 minutes, the hornworm can be drained of the hemolymph. After the hemolymph is harvested the microaggregates can be counted under a microscope. The presence of microaggregates will indicate the presence of a contaminant. To determine the quantity of the contaminants in the test sample, appropriate control groups should be utilized to establish a dose response range. This does response range can then be utilized to compare to the test results. The dose response range can be established by utilizing known quantities of a particular contaminant being tested. FIG. 1 illustrates a two-dimensional graph depicting a dose response for LPS in a tobacco hornworm system. The graph in FIG. 1 illustrates that the tobacco horn worm system is capable of detecting as little as 10 ug of LPS. Additional controls should be set up by injecting equivalent amounts of sterile water into one control group and the carrier utilized into another control group. A student's t-test can be utilized to determine if there is a significant difference between the control groups and the test group. In addition to performing the student's t-test, one skilled in the art will be able to prepare the dose response range and the control groups.

Experiments should also be performed in order to determine a minimum range of detection and efficiency for specific insect bioassay systems and samples to be tested. As previously stated, this can be accomplished by injecting a range of known standards of a contaminant into the insect of choice. After the injection, the time and number of nodules present should be recorded. These ranges should be established prior to implementation of the insect bioassay system for a particular type of sample. The data collected from these experiments will provide a standard for a particular insect bioassay system and contaminant being tested. These experiments are known to those skilled in the art.

An example of the best mode utilized for the method of the present invention is set out in Example 1.

The present invention can also be performed in vitro on hemolymph that has previously been collected from an insect. Hemolymph can be harvested from insects, preserved, and used at a later date. Through this process, experiments can be preformed to determine the presence of a contaminant without directly working with insects. The collection procedure for obtaining hemolymph is called the pericardial puncture procedure, developed by Horohov and Dunn, and described in J. Invert. Pathol., 1983, 41:203-213, which is hereby incorporated by reference.

In addition to a method for detecting and quantifying a contaminant in a sample, the present invention includes a kit, wherein the kit includes at least one aliquot of naive insect eggs, a syringe, and a protocol. The naive insect eggs can be any type of insect that undergoes microaggregate production after the injection of a contaminant. In the preferred embodiment, this insect is the tobacco homworm. The kit can also include at least one sterile syringe which can be utilized to inject the insect with the sample to be tested or solutions utilized as controls. Lastly, the kit can include a protocol. The protocol will set out procedures for the detection and quantifying of the particular insect system, proper controls to be run in combination with the sample to be tested, and a brief description of the detection method. The kit of the present invention will allow the detection and quantification of a contaminant.

In a second embodiment, the kit includes an amount of hemolymph that has not been activated by exposure to a wound during harvesting from the insect. The pericardial puncture procedure can also be utilized in the lab to collect hemolymph for in vitro experiments. The pericardial puncture procedure is known to those skilled in the art and is incorporated by reference above. This procedure avoids activating the hemolymph during the collection.

The present invention further provides an article of manufacture comprising a packaging material and the materials required to perform the method of the present invention, wherein the materials required to perform the method of the present invention comprise an amount of hemolymph. The article of manufacture can also include at least one syringe and a protocol. Alternatively, the article of manufacture will comprise at least one aliquot of naive insect eggs. The packaging material can be formed of any type of material that will contain the materials required for the method of the present invention. The packaging materials will also contain a label stating the contents and what they are to be used for. Specifically, the label will briefly describe the method and the contents. One skilled in the art would know what the packaging and label would comprise.

### EXAMPLE 1

Anesthetize the insects by chilling on ice for 15 minutes, then surface sterilize them by swabbing their surfaces with 95% ethanol. Then, inject selected contaminant, taking care to keep the needle parallel to the body wall to avoid internal injuries. A 10 μl injection is used for large insects (fifth instar M. sexta); however, smaller volumes are used for smaller insects. After preselected incubation periods, withdraw hemolymph by pericardial puncture, using 21-gauge 1.5″ needles. The pericardial puncture was designed by Horohov and Dunn (1983) to obtain hemolymph that is not activated by exposure to a wound site during the collection process. For tobacco hornworms, insects are anesthetized by chilling on ice, then surface sterilized by swabbing them with 95% EtOH. While holding the homworm with head bent downward between thumb and index finger, a 1.5″ 20-gauge needle with hub removed is inserted (bevel up) anteriorly through the dorsal cuticle at the junction of the thorax and abdomen. The needle is held parallel to the integument so the needle penetrates the pericardial sinus without damage to other tissues. Freely dripping hemolymph is collected into chilled buffer in a sterile polypropylene test tube (1.5 ml). Needles are never used for more than one insect, and hemolymph from two or more insects is not combined. Begin with a time course to select optimal incubation periods for routine experiments. Typically, 1, 2, 4, 8, 12, 18 and 24 hour incubations for the initial inquiry. Dilute the hemolymph 1:1 with chilled Grace's Insect Medium, then mix gently by inverting several times. Grace's Insect Medium is made of 0.58 g/l Pipes (1,4-piperazinediethanesulfonic acid), amended with 0.23 g/l NaCl; 2.98 g/l KCI; 3.66 g/l MgCl₂; 83.0 g/l sucrose; 1.0 g/l polyvinylpyrrolidone; 30 mg/l penicillin G; 15.0 mg/l phenylthiourea, made to pH 6.5. The buffer can be stored in the refrigerator three to four weeks. Grace's Insect Medium is routinely purchased from biological suppliers.

Apply 20 μl of dilute hemolymph to hemacytometer and view under a microscope. Count microaggregates touching the upper and right hand lines of each square; do not count microaggregates touching the left and lower lines. Use the corner grids, rather than the center grids. In the preferred method, an aggregate is defined as 10 or more cells clustered together. The calculation of Microaggregates per ml of hemolymph is computed using this formula:

microaggregates=number of aggregates×dilution factor×10⁵/number of zones counted

All references discussed herein are specifically incorporated in their entirety in all respects.

From the foregoing, it will be seen that this invention is one well-adapted to attain all those ends and objects hereinabove set forth, together with other advantages which are obvious and which are inherent to the invention. It will be understood that certain features and subcombinations are of utility and may be employed without reference to other features and subcombinations. This is contemplated by and is within the scope of the claims. Since many possible embodiments may be made of the invention without departing from the scope thereof, it is to be understood that all matters herein set forth are to be interpreted as illustrative and not in a limiting sense.

We claim:

1. A kit comprising:
a) at least one aliquot of naive insect eggs; and
b) at least one syringe.

2. The kit of claim 1 wherein the at least one aliquot of naive insect eggs are from a tobacco hornworm.

3. The kit of claim 1 further comprising a protocol.

4. An article of manufacture comprising a packaging material and at least one aliquot of naive insect eggs, wherein the packaging material comprises a label that indicates that the aliquot of naive insect eggs can be used to detect a contaminant in a sample.

5. The article of manufacture of claim 4 wherein the article of manufacture further comprises a protocol.

6. The article of manufacture of claim 4 wherein the article of manufacture further comprises a syringe.

7. The article of manufacture of claim 4 wherein the aliquot of naive insect eggs is from a tobacco hornworm.

8. An article of manufacture comprising a packaging material and at least one aliquot of hemolymph wherein the packaging material comprises a label that indicates that the at least one aliquot of hemolymph can be used to detect a contaminant in a sample.

9. The article of manufacture of claim 8 wherein the article of manufacture further includes a protocol.

10. The article of manufacture of claim 8 wherein the article of manufacture further includes a syringe.

11. An article of manufacture comprising a packaging material and at least one insect wherein the packaging material comprises a label that indicates that the insect can be used to detect a contaminant in a sample.

12. The article of claim 11 wherein the insect is a tobacco hornworm.
